(19) United States
(12) Patent Application Publication
(10) Pub. No.: US 2024/0230122 A9
(43) Pub. Date: Jul. 11, 2024

(54) HVAC OIL DIFFUSER SYSTEM

(71) Applicant: JAMES GHORMLEY, Arlington, TX (US)

(72) Inventor: JAMES GHORMLEY, Arlington, TX (US)

(21) Appl. No.: 18/048,823

(22) Filed: Oct. 21, 2022

(51) Int. Cl. F24F 8/50; A61L 9/14

(52) U.S. Cl. CPC F24F 8/50; A61L 9/14; A61L 2209/111; A61L 2209/12; A61L 2209/133; A61L 2209/134

(57) ABSTRACT

A liquid diffuser system includes a housing with one or more electrical components configured to selectively release a singular air mixture from multiple atomized sources for dispersing throughout multiple individually distinct and separate rooms simultaneously. The system includes an air pump to collect and pressurize air for passage into multiple bottles, each of which contain a liquid for atomization and mixture with the air. A splitter is used to combine the air from each bottle into a singular air mixture for releasing to the environment. The system may also include an air sensor located within a duct of an HVAC system for dispersing the air into the duct work. The liquid diffuser system may include the HVAC system and duct work. The system may also be regulated through a remote electronic device.

### BACKGROUND OF THE INVENTION

### 1. Field of the Invention

The present application relates to an oil diffuser system, and more particularly to an oil diffuser system integrated with an HVAC system to allow multiple fragrances and control via a remote electronic device.

### 2. Description of Related Art

Diffusers are used to diffuse essential oils into the air by one or more processes. The diffused oil may provide a desirable fragrance. Processes may include evaporative processes, heat applications, a nebulizer, and ultrasonic diffusion. These are commonly seen as plug in devices that have a small housing hardly bigger than an outlet cover wherein they plug into the wall and slowly emit a scent to the air. In more complex systems, some automated controls are possible. These tend to have larger housings and can be regulated by turning it on or off, or even be scheduled.

A downside to the systems presently in the market is that they are limited to a singular room and typically a singular fragrance at a time. To swap fragrances, a user would interchange the oil cartridge with another cartridge of a different oil. To reach multiple rooms a user would have to purchase multiple units and regulate them each individually.

Efforts to provide a diffuser for an HVAC setting have been limited to embodiments using industrial steel housings with single scent options from the oil and a failure to allow customizable control through remote devices. Generally, these systems sit within a room to provide fragrance over a wide area only. These are larger than the small wall plug-in types on the wall outlet. Although strides have been made, shortcomings remain.

### BRIEF SUMMARY OF THE INVENTION

It is an object of the present application to provide an oil diffuser system configured to provide a user multiple fragrance options, air flow detection to assist with the feature of an auto-run mode, oil level detection to know when oil bottles are running low, motion detection to improve user experience and make the system more efficient, as well as mobile app technology to permit remote regulation of the system from a remote electronic device.

It is another object of the present application to provide a system using the oil diffuser system inclusive with an HVAC system of a structure and a remote electronic device. The oil diffuser system is in communication with the ducting of the HVAC system wherein an air sensor is configured to monitor the flow of air through the duct work. The system is configured to release an air mixture into the duct work when airflow is detected so as to emit that air mixture throughout a structure. It is an object of the present application that the combined system allows the air mixture to reach independent rooms in the structure. A person is able to use software applications from a remote electronic device to regulate performance.

Ultimately the invention may take many embodiments. In these ways, the present invention overcomes the disadvantages inherent in the prior art. The more important features have thus been outlined in order that the more detailed description that follows may be better understood and to ensure that the present contribution to the art is appreciated. Additional features will be described hereinafter and will form the subject matter of the claims that follow.

Many objects of the present application will appear from the following description and appended claims, reference being made to the accompanying drawings forming a part of this specification wherein like reference characters designate corresponding parts in the several views.

Before explaining at least one embodiment of the present invention in detail, it is to be understood that the embodiments are not limited in its application to the details of construction and the arrangements of the components set forth in the following description or illustrated in the drawings. The embodiments are capable of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the various purposes of the present design. It is important, therefore, that the claims be regarded as including such equivalent constructions insofar as they do not depart from the spirit and scope of the present application.

### BRIEF DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a schematic of an oil diffuser system according to an embodiment of the present application.

FIG. 2 is a perspective view of the oil diffuser system of FIG. 1.

FIG. 3 is a lower rear perspective view of the oil diffuser system of FIG. 2.

FIG. 4 is a front view of the oil diffuser system of FIG. 2.

FIG. 5 is a rear view of the oil diffuser system of FIG. 2.

FIG. 6 is a right view of the oil diffuser system of FIG. 2.

FIG. 7 is a left view of the oil diffuser system of FIG. 2.

FIG. 8 is a bottom view of the oil diffuser system of FIG. 2.

FIG. 9 is a top view of the oil diffuser system of FIG. 2.

FIG. 10 is an alternate front view of the oil diffuser system of FIG. 4 with a front cover removed.

FIG. 11 is an enlarged section view of the oil diffuser system of FIG. 7.

FIG. 12 is a schematic of an exemplary electronic device used with the oil diffuser system of FIG. 1.

While the embodiments and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE INVENTION

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the embodiments described herein may be oriented in any desired direction.

The embodiments and method in accordance with the present application overcomes one or more of the above-discussed problems commonly associated with the prior art discussed previously. In particular, the oil diffuser system of the present application is configured to allow for the attachment and operation of multiple fragrance oils simultaneously. The system is configured to detect air flow so as to regulate operation in an auto-run mode. Oil level sensors are included to monitor oil level in the oil bottles. A number of LED lights are included to enhance user experience as well as a motion sensor. The system as a whole is operable through a mobile app loT control program. These and other unique features are discussed below and illustrated in the accompanying drawings.

The embodiments and method will be understood, both as to its structure and operation, from the accompanying drawings, taken in conjunction with the accompanying description. Several embodiments of the assembly may be presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

Referring now to the Figures wherein like reference characters identify corresponding or similar elements in form and function throughout the several views. The following Figures describe embodiments of the present application and its associated features. With reference now to the Figures, embodiments of the present application are herein described. It should be noted that the articles “a”, “an”, and “the”, as used in this specification, include plural referents unless the content clearly dictates otherwise.

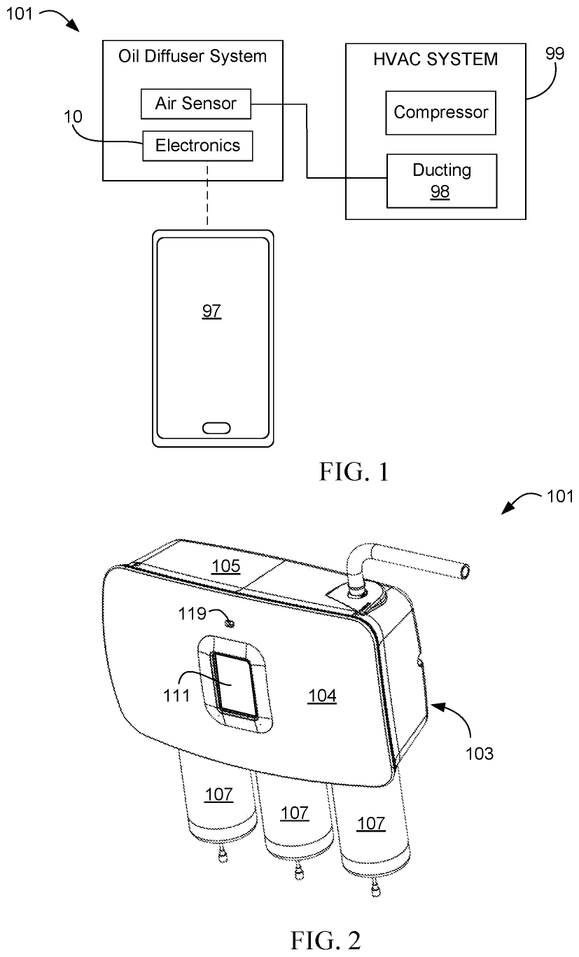

Referring now to FIG. 1 in the drawings, a schematic of system 101 is illustrated in combination with additional components that may optionally be part of system 101 as a whole. Shown in FIG. 1 is oil diffuser system 101 as a discrete and individual device. System 101 is configured to include an air sensor (see air sensor 124 below) in communication with a duct 98 of an HVAC system 99. System 101 is configured to detect airflow through duct 98 and selectively disperse an air mixture into the duct 98 for distribution through the duct work to all areas that duct work is routed. This allows one system to be able to disperse an air mixture through multiple independent spaces efficiently and effectively. A user may use an electronic device 97 to regulate performance of system 101 through one or more software applications. Together as a whole system 101 may include the HVAC system 99, or any portions thereof, and electronic device 97. Alternatively, system 101 may be thought of as an independent unit. For explanatory purposes herein, system 101 will be described as its own unit with communication to HVAC 99 and device 97

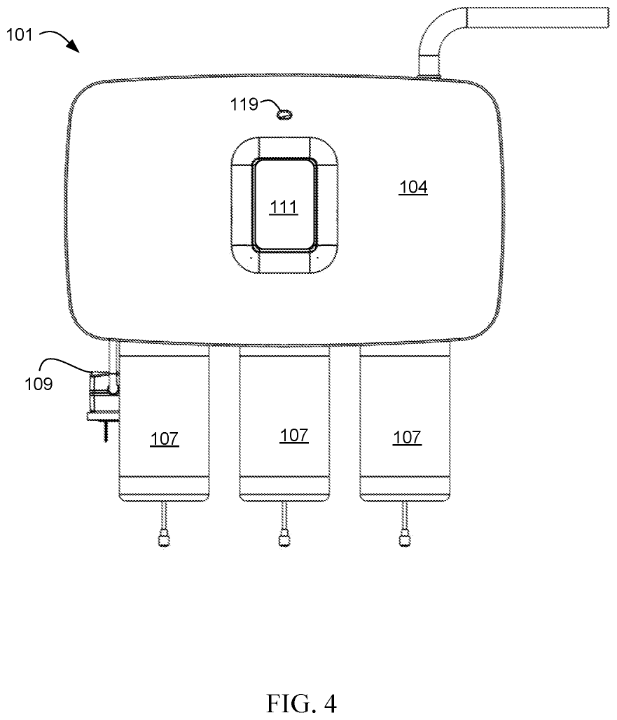

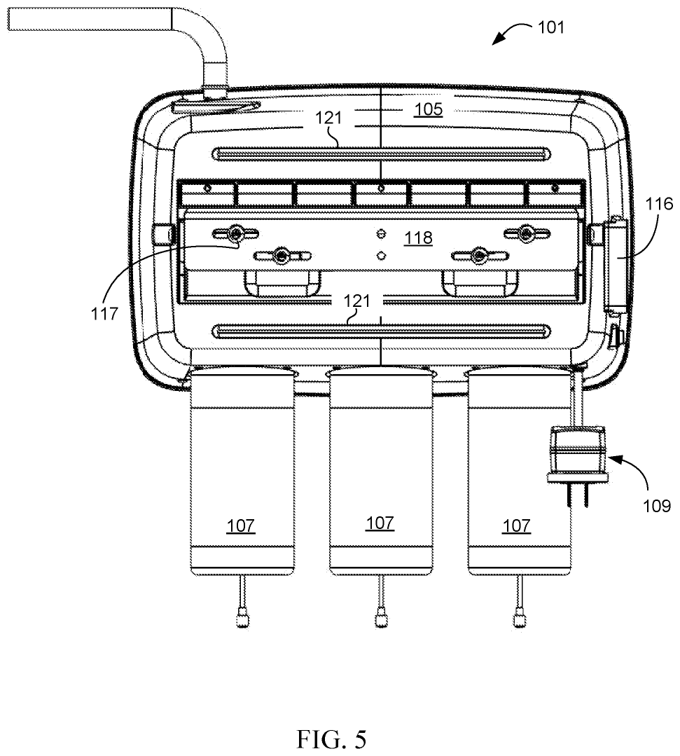

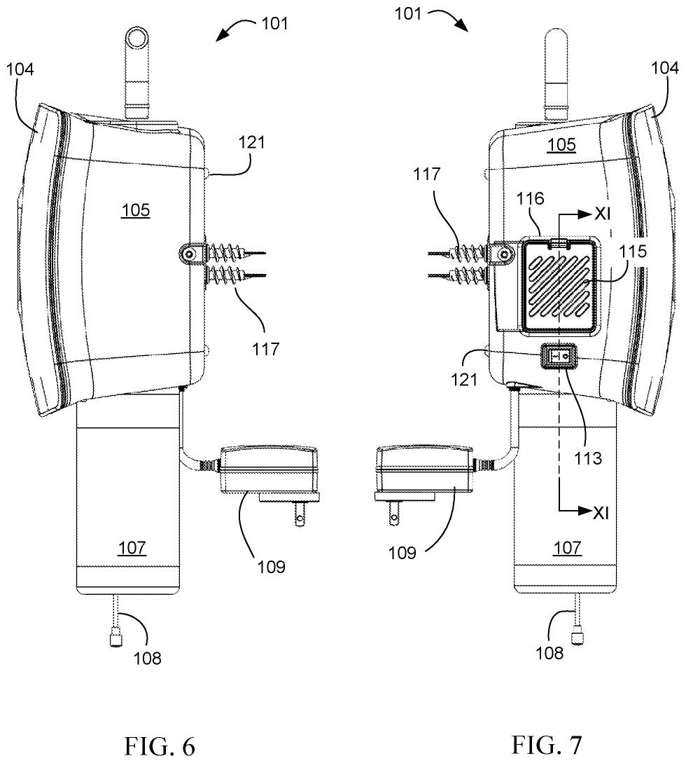

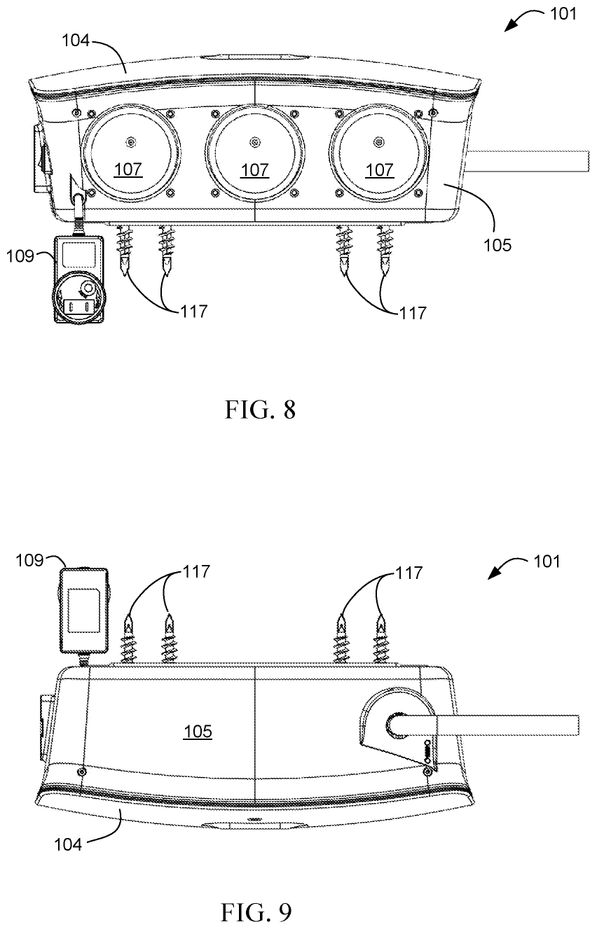

Referring now to FIGS. 2-9 in the drawings, various views of an HVAC oil diffuser system 101 is provided. System 101 is configured to include a housing 103 having a front cover 104 and a rear body 105. System 101 include multiple components individually situated within housing 103. A plurality of oil bottles 107 are coupled to and/or through housing 103 along a lower surface of rear body 105. It is understood that one or more oil bottles 107 may be used and that the number depicted is exemplary in nature. More or less oil bottles 107 may be incorporated into the design.

Oil bottles 107 are configured to hold a volume of fluid. Although termed herein as “oil” bottles 107, bottles 107 may be configured to hold any fluid, such as water, essential oils, medication, any solution of liquid, and so forth. Bottles 107 are secured to housing 103 in any known method, such as through interference fit with threaded connections for example. It is important that whatever connection is used, that such connection allows for a seal to prevent leaks. Extending down below and passing through to the interior portion of bottles 107 are wicks 108. Each bottle 107 has at least one wick 108. The exact length of wick 108 is not limited to that shown herein.

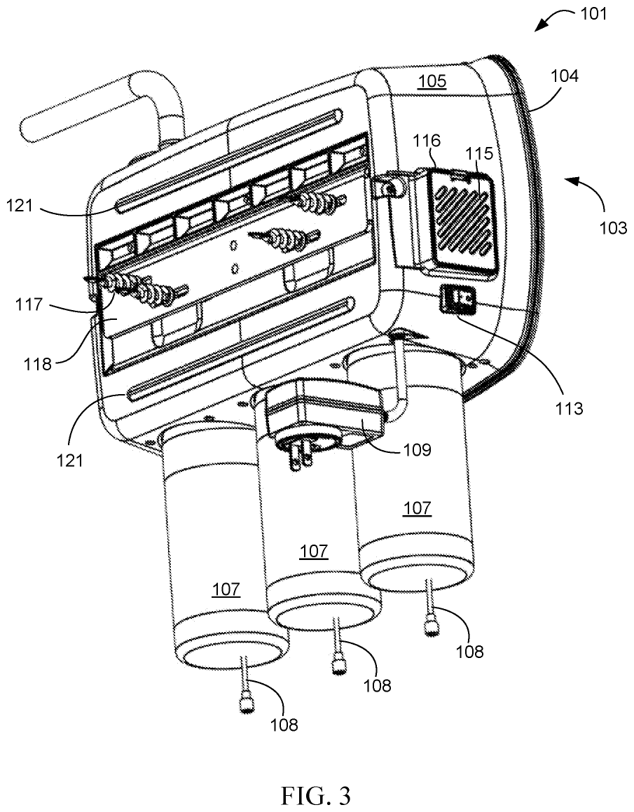

Also included in system 101 is a power adapter 109 to allow system 101 to receive power from an electrical source, such as the power grid or a generator for example. Power adapter 109 is more clearly visibly in FIGS. 3 and 5-7 in the drawings. Power adapter 109 may take other shapes apart from that depicted. Power received through power adapter 109 provides electricity to the electrical components of system 101. One such component is touchscreen 111 configured to provide a user interface for the regulation, monitoring, and maintenance of system 101. Switch 113 is coupled to housing 103 and is configured to turn on and off system 101.

Also coupled to housing 103 is filter 115. Air is pulled from the environment through filter 115 and is used to mix with vaporized liquids through bottles 107 before being released subsequently to back to the environment. Filter 115 includes a cover 116 and is interchangeable or replaceable to allow for a clean filter to be used at all times. Filter 115 may be changed without having to open housing 103, but by merely removing the cover external to housing 103.

As noted from the Figures, mounting hardware 117 is also included to permit system 101 to be affixed to a wall or vertical mounting surface. In the Figures are shown sheetrock anchors but any other fastener may be used. Hardware 117 is optional in that they may be removed. However, with bottles 107 located along the lower surface of housing 103, it is ideally acceptable to use hardware 117. A user may mount system 101 to any location in a room. An ideal location would be one where good airflow and visibility of the room is provided. A mounting plate 118 is used to reinforce rear body 105

With respect to visibility, system 101 is configured to include a motion sensor 119 coupled to front cover 104. Sensor 119 is configured in a manner within system 101 to allow it to have unobstructed view in front of housing 103. It is understood that the particular location may vary in some embodiments and should not be restricted to this particular location. Motion sensor 119 is configured to detect movement in the room in which it is located. System 101 may be programmed with a sleep mode which turns off when no movement is detected. When movement is detected, system 101 may the be programmed to run in an active mode in accordance with preset user commands. Sensor 119 helps to make system 101 more efficient and conserve the liquids in bottles 107 for when users are around for example. This setting of a “sleep mode” may or may not be used or incorporated by the user but is an option that may exist.

Also seen in the Figures, one or more lights 121 may be coupled to housing 103. Light 121 is configured to selectively illuminate. Light 121 may be used to enhance the aesthetic appeal to a user. In some situations, perhaps light 121 is an indication to the user of the current operating status of system 101. For example, when light 121 is off, system 101 may be in a sleep mode or turned off. When light 121 is on, system 101 may be in an operating mode. It is understood that light 121 may illuminate one or more colors. This feature is used to allow customization by the users. An example could be where a user may wish to illuminate a color that matches the décor of the room. A user may use touchscreen 111 to regulate color selection and even brightness. Other lighting features may be regulated as well, such as timing. As seen in the Figures, light 121 is coupled to rear body 105 but may be located in any other face, surface, or portion of housing 103 to provide an external illumination of light. Light 121 may be a light bar for example as shown.

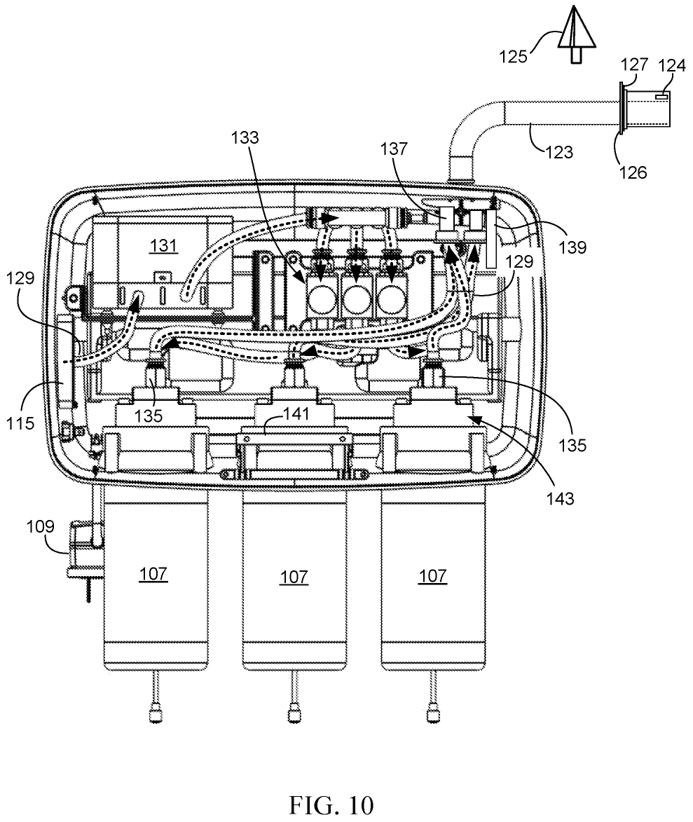

Referring now also to FIG. 10 in the drawings, a front view of system 101 is provided wherein front cover 104 is removed. Filter 115 is shown partially extending into the interior of housing 103. Internal tubing 129 is shown extending between a number of components. In operation, air is pulled through filter 115 and through tubing 129 to air pump 131. Air is pressurized and passed through additional tubing 129 to a set of solenoids 133 which is configured to regulate the passage of air to one or more bottles 107. The air may be regulated in terms of air flow wherein air flows into respective bottles may be different. Additionally, air flow into only one bottle 107 may be permitted. Likewise, air flow into two bottles 107 may be allowed wherein they have equal air flow between them. From solenoids 133, air is selectively passed through tube fittings 135 into bottle 107. Each bottle has tube fittings 135. Tube fittings 135 include an inlet for the air to pass and an exit wherein the atomized or mixed air is released out through additional tubing to a splitter fitting 137 configured to introduce the respective air from each bottle 107 together before passage through outer tubing 123.

External tubing 123 extends out an opening in housing 103 configured to hold a combined air and liquid mixture for releasing back into the environment. Tubing 123 may be any length necessary to fully locate air sensor 124 in a desired location. A seal 127 and grommet 126 may be used to help seal air sensor 124 in its location. A drill bit 125 is also optionally included to assist in installation. One of the many features of system 101 is that it is configured to be integrated into the HVAC system of a structure or building to permit for the release of the combined air mixture throughout multiple rooms at once using the air from the existing HVAC system.

Air sensor 124 is configured to be located within at least one air duct 98 of an HVAC system. Air released through tubing 123 is passed into the duct 98 and distributed to the various rooms to which that ducting is connected. This feature allows a single oil diffuser system to permit the distribution of air over a vastly larger area and throughout individually segregated spaces. With existing technology, a user would have to have multiple individual systems with multiple cannisters of fluid and regulate them all individually. The conventional methods are time consuming, expensive, and tedious on a user to say the least.

It should be noted that solenoid 133 and splitter 137 work together to regulate the exact air mixture distributed. In operation, air sensor 124 is configured to monitor the flow of air in duct 98, when detected, system 101 is configured to release a quantity of mixed air into duct 98 for distribution. This usage of duct 98 helps the air mixture more evenly distribute not only through a room, but through a whole building.

Further with respect to FIG. 10, an oil sensor cable 139 is in communication with splitter 137 and is configured to detect and/or regulate oil in system 101. Furthermore, a panel hinge 141 is shown and is configured to help secure bottles 107 within housing 103. The bottles 107 are configured to engage a bottle mount assembly 143.

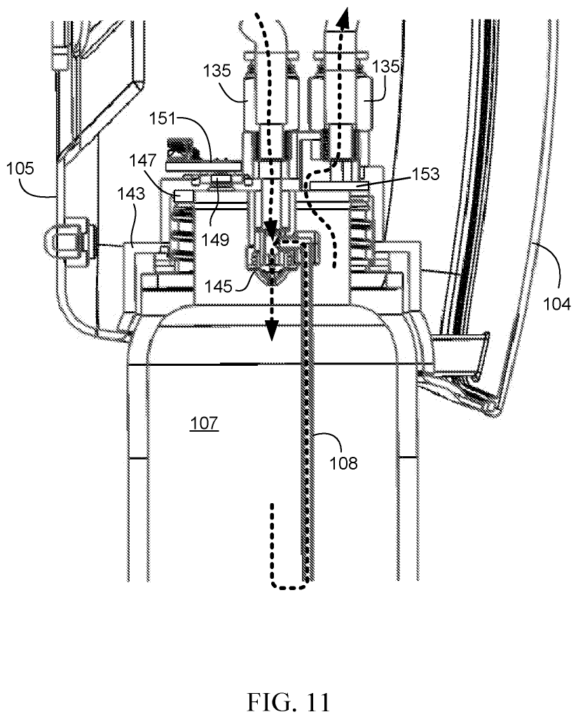

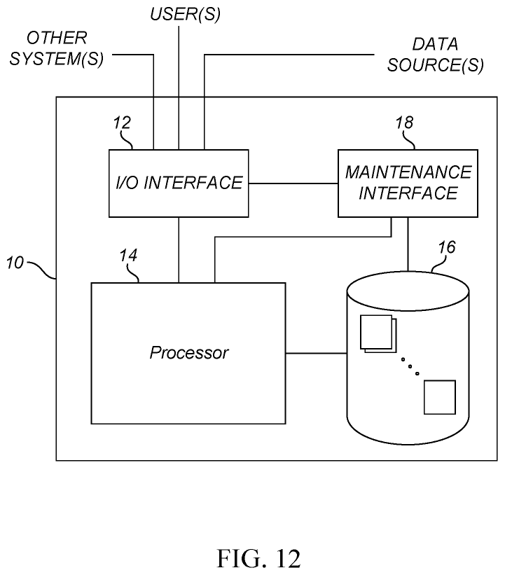

Referring now also to FIG. 11 in the drawings, an enlarged side section view of system 101 in FIG. 7 is illustrated. This view is useful to show the flow of air adjacent bottles 107 through tube fittings 135. As seen, one tube fitting 135 allows entrance of air from solenoids 133. Another tube fitting 135 allows for the release of air from bottle 107 to splitter 137. Air that enters bottle 107 is passed through nozzle 145 where it is atomized with air that comes from bottle 107 and is passed through wick 108. Air is then selectively released through tube fitting 135 to go to splitter 137.

This Figure is useful to see bottle 107 seated in bottle mount assembly 143. A gasket 147 seals bottle 107 to mount assembly 143. A lens and gasket assembly 149 is also shown atop bottle mount assembly 143. Fluid level sensor 151 is configured to measure the level of fluid in bottle 107 by “seeing” through the lens. Another component in FIG. 11 is the use of a cover 153 configured to selectively close off the exit passage of air from bottle 107.

Referring now also to FIG. 12 in the drawings, a schematic of an electronic device is illustrated. Electronic device 10 is illustrative of the circuitry and programing, both hardware and software related, of system 101 used to monitor, process, execute, and operate in the manner described herein. Electronic device 10 may be partially integrated within housing 103 and/or also part of a broader network including one or more remote electronic devices. System 101 may be operational with one or more remote electronic devices through the input/output interface and is operational with both wired and/or wireless communications.

The electronic device 10 includes an input/output (I/O) interface 12, a processor 14, a database 16, and a maintenance interface 18. Alternative embodiments can combine or distribute the input/output (I/O) interface 12, processor 14, database 16, and maintenance interface 18 as desired. Embodiments of the electronic device 10 can include one or more computers that include one or more processors and memories configured for performing tasks described herein below. This can include, for example, a computer having a central processing unit (CPU) and non-volatile memory that stores software instructions for instructing the CPU to perform at least some of the tasks described herein. This can also include, for example, two or more computers that are in communication via a computer network, where one or more of the computers includes a CPU and non-volatile memory, and one or more of the computer's non-volatile memory stores software instructions for instructing any of the CPU(s) to perform any of the tasks described herein. Thus, while the exemplary embodiment is described in terms of a discrete machine, it should be appreciated that this description is non-limiting, and that the present description applies equally to numerous other arrangements involving one or more machines performing tasks distributed in any way among the one or more machines. It should also be appreciated that such machines need not be dedicated to performing tasks described herein, but instead can be multi-purpose machines, for example computer workstations, that are suitable for also performing other tasks. Furthermore, the computers may use transitory and non-transitory forms of computer-readable media. Non-transitory computer-readable media is to be interpreted to comprise all computer-readable media, with the sole exception of being a transitory, propagating signal.

The I/O interface 12 provides a communication link between external users, systems, and data sources and components of the electronic device 10. The I/O interface 12 can be configured for allowing one or more users to input information to the electronic device 10 via any known input device. Examples can include a keyboard, mouse, touch screen, microphone, and/or any other desired input device. The I/O interface 12 can be configured for allowing one or more users to receive information output from the electronic device 10 via any known output device. Examples can include a display monitor, a printer, a speaker, touchscreen 111, and/or any other desired output device. The I/O interface 12 can be configured for allowing other systems to communicate with the electronic device 10. For example, the I/O interface 12 can allow one or more remote computer(s) to access information, input information, and/or remotely instruct the electronic device 10 to perform one or more of the tasks described herein. The I/O interface 12 can be configured for allowing communication with one or more remote data sources. For example, the I/O interface 12 can allow one or more remote data source(s) to access information, input information, and/or remotely instruct the electronic device 10 to perform one or more of the tasks described herein.

The database 16 provides persistent data storage for electronic device 10. While the term “database” is primarily used, a memory or other suitable data storage arrangement may provide the functionality of the database 16. In alternative embodiments, the database 16 can be integral to or separate from the electronic device 10 and can operate on one or more computers. The database 16 preferably provides non-volatile data storage for any information suitable to support the operation of the electronic device 10, including various types of data discussed herein.

The maintenance interface 18 is configured to allow users to maintain desired operation of the electronic device 10. In some embodiments, the maintenance interface 18 can be configured to allow for reviewing and/or revising the data stored in the database 16 and/or performing any suitable administrative tasks commonly associated with database management. This can include, for example, updating database management software, revising security settings, and/or performing data backup operations. In some embodiments, the maintenance interface 18 can be configured to allow for maintenance of the processor 14 and/or the I/O interface 12. This can include, for example, software updates and/or administrative tasks such as security management and/or adjustment of certain settings.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

What is claimed is:

1. A liquid diffuser system, comprising:
a housing;
a first bottle coupled to the housing;
an air pump within the housing configured to draw air from the environment;
a solenoid configured to regulate the passage of air from the air pump;
a nozzle located within the first bottle configured to atomize the incoming air with a liquid in the first bottle to generate an air mixture; and
an external tube extending away from the housing for the release of the air mixture.

2. The system of claim 1, further comprising:
an air filter coupled to a portion of the housing so as to filter out contaminants from the air prior to the air pump.

3. The system of claim 1, further comprising:
a second bottle coupled to the housing adjacent the first bottle, the second bottle also containing a liquid, the second bottle in communication with the air pump to receive air from the environment; and
a splitter configured to receive an air mixture from both the first bottle and the second bottle and selectively combine them into a singular mixture.

4. The system of claim 1, further comprising:
a touchscreen coupled an outer portion of the housing and configured to let a user regulate performance of the system.

5. The system of claim 1, further comprising:
a motion sensor configured to monitor for movement in the environment.

6. The system of claim 1, further comprising:
a light coupled to an exterior of the housing configured to emit a light.

7. The system of claim 6, wherein the light is configured to emit different colors.

8. The system of claim 7, wherein the colors of the light are representative of an operational mode of the system.

9. The system of claim 1, further comprising:
an air sensor in communication with the external tube, the air sensor configured to monitor air flow adjacent the external tube, wherein the detection of an air flow initiates an operational function of the system.

10. The system of claim 9, further comprising:
an HVAC system containing a duct;
wherein the air sensor is located within the duct to detect the air flow passing within the duct.

11. The system of claim 1, further comprising:
a remote electronic device in communication with the system and configured to regulate operational functions of the system remotely.

12. A liquid diffuser system, comprising:
a housing;
a first bottle coupled to the housing;
a second bottle coupled to the housing adjacent the first bottle;
an air pump within the housing configured to draw the air from the environment;
a solenoid configured to regulate the passage of air from the air pump;
a pair of nozzles individually located within the first bottle and the second bottle, the pair of nozzles are configured to atomize the incoming air with a liquid in the first bottle to generate an air mixture;
a splitter configured to receive the air mixture from both the first bottle and the second bottle and selectively combine them into a singular air mixture;
an external tube extending away from the housing for the release of the singular air mixture; and
an air sensor in communication with the external tube, the air sensor configured to monitor air flow adjacent the external tube, wherein the detection of an air flow outside of the external tube initiates an operational function of the system.

13. The system of claim 12, further comprising:
an HVAC system containing a duct;
wherein the air sensor is located within the duct to detect the air flow passing within the duct.

14. The system of claim 12, further comprising:
a remote electronic device in communication with the system and configured to regulate operational functions of the system remotely.

15. The system of claim 12, wherein the air mixture is configured to be released into multiple separate rooms.

16. The system of claim 1, further comprising:
a light coupled to an exterior of the housing configured to emit a light.

17. The system of claim 16, wherein the light is configured to emit different colors.

18. The system of claim 17, wherein the colors of the light are representative of an operational mode of the system.

19. The system of claim 12, further comprising:
a motion sensor configured to monitor for movement in the environment.
